(19) United States
(12) Patent Application Publication
(10) Pub. No.: US 2013/0317286 A1
(43) Pub. Date: Nov. 28, 2013

(54) ANTI-EROSION SORFT TISSUE REPAIR DEVICE

(71) Applicant: BVW Holding AG, (US)

(72) Inventors: Lukas Bluecher, Eurasberg (DE); Michael Milbocker, Holliston, MA (US)

(21) Appl. No.: 13/886,809

(22) Filed: May 3, 2013

Related U.S. Application Data
(63) Continuation-in-part of application No. PCT/US2013/027345, filed on Feb. 22, 2013.
(60) Provisional application No. 61/603,008, filed on Feb. 24, 2012.

(51) Int. Cl. A61F 2/00

(52) U.S. Cl. CPC A61F 2/0063
USPC 600/37

(57) ABSTRACT

The present disclosure relates to surgical implants comprising a bioabsorbable incision reinforcement element, a long-term mesh, and a bioabsorbable coating disposed on the mesh. The surgical implants disclosed herein are useful in a variety of surgical procedures, particularly surgeries involving the pelvic floor. More particularly, the present disclosure relates to surgical implants, wherein an incision reinforcement element comprises a bioabsorbable material that degrades during a first time period, and the coating comprises a bioabsorbable material that degrades in a second time period, and the first time period is shorter than the second time period.

### CROSS-REFERENCE TO RELATED APPLICATIONS

This application is a continuation-in-part of International Application No. PCT/US2013/027345, filed on Feb. 22, 2013, which claims the benefit of priority to U.S. Provisional Application No. 61/603,008, filed on Feb. 24, 2012, both of which are hereby incorporated by reference in their entirety.

### TECHNICAL FIELD

The present disclosure relates to implantable medical devices that are suitable for use in a variety of surgical procedures, including but not limited to pelvic floor surgeries, such as pelvic organ prolapse.

### BACKGROUND

Pelvic organ prolapse is a descending of pelvic organs (bladder and/or uterus and/or rectum) from their normal position when they tend to protrude through the vulvo-vaginal opening. This phenomenon results from weakening of the supportive, cohesive and organ-suspending systems. Three visceral compartments of the pelvis may be concerned: anterior compartment (urinary), middle compartment (genital) and posterior compartment (digestive). All three of these aspects are served individually by the present disclosure, as well as combinations of these aspects, as is typically encountered clinically.

The maintenance of normal pelvic-perineal anatomy is based on these three systems having synergistic action. Hence, it would be useful to provide an implantable device that can augment this tri-functional arrangement. For example, an implant should suspend in a manner analogous to ligaments. An implant also should provide a cohesive system in the manner analogous to connective tissue bridging different organs. An implant should further provide support analogous to the levator ani muscle joining between the vulva and the anus to form the perineal central fibrous core. These are structural features that would be useful in implants, but there is also a temporal aspect to implants, wherein the body starts to replace the function of the implant by natural structures. Therefore, providing the correct choreography between synthetic and living structures would be useful in preventing erosion, or rejection of an implant.

Intra-abdominal pressure forces are isotropic and are oriented towards the posterior perineum and sacral cavity, preserving the weak point formed by the uro-genital slit. When pelvic statics are perturbed, the resultant pressure forces places demand on the uro-genital slit. This condition may persist without anatomical repair; hence, a permanent repair in many cases is required.

Pelvic floor disorders include cystocele, rectocele, enterocele and uterine and vaginal vault prolapse. These disorders typically result from weakness or damage to normal pelvic support systems. The most common etiologies include childbearing, removal of the uterus, connective tissue defects, prolonged heavy physical labor and postmenopausal atrophy. Vaginal vault prolapse is the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons.

Vaginal vault prolapse is often associated with a rectocele, cystocele or enterocele. Accordingly, a multi-variant approach is prescribed. It is known that in order to repair vaginal vault prolapse by suturing to the supraspinous ligament or to attach the vaginal vault through mesh or fascia to the sacrum is extreme, and does not allow the body to remodel the support in a natural way. As a consequence, patients suffering from vaginal vault prolapse may also require a surgical procedure to correct stress urinary incontinence that is either symptomatic or latent. Thus, there is a need for an implant that provides a first high strength fixed structure and later remodels with the tissue to provide a more flexible construct.

It is reported that 72% of patients with vault prolapse had a combination of other pelvic floor defects. See Richter K: Massive Eversion of the Vagina: Pathogenesis, Diagnosis and Therapy of the True Prolapse of the Vaginal Stump, Clin. Obstet Gynecol 25:897-912 (1982). Unfortunately, the state of current implants limits surgeons to a bimodal approach—either permanent or absorbable. The absorbable implants, either biologic or synthetic, lose strength quickly, whereas the permanent implants create long term inflammatory responses that actually contribute to erosion.

The high failure rate for prolapse repair has motivated the development of a number of implants to augment the known prolapse repair methods. Most of them are misdirected in the sense that they actually augment foreign body response, which is at the root of erosion. In particular, the use of biologics comprise a two-fold disadvantage in that they augment foreign body response and provide minimal structure support. It is generally understood that removing a synthetic implant from functional tissue, by absorption, is beneficial. It should also be understood that such tissue needs to be directed in its reconstruction, and a phased absorbable approach achieves this end.

Mesh repair systems have proven especially beneficial for women who have weak tissues that otherwise would not support conventional prolapse repair. While mesh systems provide many benefits, long term results suggest an unacceptable rate of erosion of tissue, which can cause additional complications and require subsequent surgical procedures and hospitalization. The reason for the propensity for erosion is that women who benefit from prosthetic repair suffer an chronic deficiency, either due to peri-trauma or from the effects of age. In either case, a temporally staged approached to repair is clearly indicated, with the endpoint being minimal persistent implant volume.

Because implants currently are designed to illicite a robust healing response by being insulting, requiring eosinophils, histiocytes, nucleocytes_and the like, erosion rates are reported to be between 10-20% (Watson, J. Am. Coll. Surg., vol. 183, p. 257 (1996)).

One approach to this problem is to incorporate biologic material, for example to prevent erosion of tissue. Mesh produced by medical device manufacturers have introduced dermis-based systems comprising human (allograft) or animal (xenograft) dermis, such as REPLIFORM (cadaver dermis) and XENFORM (bovine dermis), both from Boston Scientific. These dermis based-systems are typically affixed to one or more ligaments to provide support to the pelvic organs. While the dermis-based systems have lower rates of erosion, the dermis systems are not as simple to secure as the mesh-based systems, and often requiring a larger dissection plane. Additionally, the dermis-based systems critically do not offer chronic support for ingrown tissues because the dermis material degrades relatively rapidly in the body. When it is crosslinked to make it more durable in vivo, it loses many of the benefits of such materials, and elicits a foreign body response more like that evoked by synthetics.

The observation that many women who undergo surgery to repair prolapsed organs experience re-prolapse, pain, or infection, suggest a basic bio-incompatibility between the implant (either structurally or materially) and the particular region of anatomy. This experience strongly suggests that the initial clinical demands differ from the long-term requirements for tissue growth and structural support. In particular, the goal should be not to replace tissue with synthetic alternatives permanently, but rather to provide a minimal reinforcement scaffold and the majority of the initial implant volume should be absorbable. In particular, emphasis should be placed on regaining a normal geometry, whereby the natural reparative processes perform optimally and naturally and the implant is mostly absorbed by the body. Thus, repair of tissue is both acute and chronic, in the normal case the magnitude of the chronic support aspect is less than the acute support aspect. Generally, a light chronic support structure with minimal foreign body response would be beneficial.

Consequently, the present disclosure recognizes that a robust structural support is needed initially after the surgery. That structural support, primarily because it is robust, restricts normal anatomical motion, and thus serves as a cast. That aspect should be removed over time as healing progresses. Current methods use a strong mesh as a soft tissue reinforcing implant, but these meshes have long and significant foreign body responses, and are not required long term. Thus, the goal should be to restore all tissue to a high metabolic rate, and this goal includes reducing the amount of dense fibrosis. These current implants have a zero metabolic rate, and therefore represent an impediment to complete healing. Thus, it would be useful to provide a support structure comprising an absorbable material that reduces in stages over time along with a final support structure that is minimal and relatively permanent.

Accordingly, the present disclosure provides implants that are useful in surgical procedures, particularly those directed to pelvic floor disorders. In particular, the present disclosure provides implants that have a variable absorption profile, such that a first element has high-strength and is fast absorbing, a second element with a slower absorption profile is provided, and a third element provides a permanent or near permanent flexible matrix to provide long term support to the surgical area.

### BRIEF SUMMARY

It is one object of the present disclosure to provide a soft tissue repair implant device comprising a fast absorbing strength element, and a slow absorbing coating on a non-absorbing mesh.

It is a further object to provide an implant that reinforces a region of a surgical incision, and said reinforcement element is absorbed in vivo around the time the incision is expected to heal.

In some embodiments the disclosure provides a surgical implant comprising a bioabsorbable incision reinforcement element, a long-term mesh, and a bioabsorbable coating disposed on the mesh. In some embodiments, the incision reinforcement element comprises a bioabsorbable material that degrades during a first time period, and the coating comprising a bioabsorbable material that degrades in a second time period, and the first time period is shorter than the second time period.

It is a further object to provide an implant comprising a long-term mesh having an ultra-low areal mass density (for example, less than about 30 g/m², such as between about 1 and about 30 g/m², or between about 1 and about 15 g/m²), coated with an absorbable biocompatible coating. The coating is designed to reduce the foreign body response during the healing period in which inflammation can lead to erosion. The coating also provides a temporary strengthening aspect to the underlying mesh, which provides additional support to the soft tissue repair while the long-term mesh is being incorporated into the wound site.

In certain embodiments, the surgical implant described herein provides a temporally staged treatment profile, wherein the implant is maximally stiff initially after implantation to support both the wound or prolapse and the surgical incision site. Subsequently, the strengthening region near the surgical incision is absorbed leaving a long-term mesh structure with a biocompatible and absorbable coating. This coating persists for the time required to fully incorporate the mesh by tissue ingrowth. Subsequent to tissue ingrowth, the coating is absorbed and a very fine long-term mesh structure remains to supplement structurally the tissue repair, which can be chronically compromised, especially in patients with collagen deficiency.

It is further an object to provide an implant comprising a mesh and a planar or sheet-like incision reinforcement element, wherein the planar or sheet-like incision reinforcement element is glued, tied, melted or otherwise attached to a side of the mesh structure, such that one side of the mesh structure allows tissue ingrowth and the other side does not. In some embodiments, the planar element is cast within the openings of the mesh and lies substantially coplanar with the mesh.

It is a further object to provide a surgical implant having spatially variable distensibility, wherein the distensibility changes temporally, wherein an incision reinforcement element underlying an incision line has essentially no distensibility, but this feature changes relatively quickly due to absorption after implantation. An absorbable coating, which may extend over the majority of the implant, prevents distention during the time of tissue ingrowth, but after absorption of the incision reinforcement element. This coating is preferably absorbable with a duration exceeding the duration of the incision strengthening element. Lastly, when first and second absorbable components are absorbed, the remaining mesh may be constructed to be more distensible in one direction than another. Typically this anisotropy is orthogonal, but it may lie on any combination of angles, and may include more than 2 characteristic axes.

It is a further object to provide a tissue reinforcing prosthetic which has minimal distensibility in a line perpendicular and planar to the incision in at least one of the aspects of a) the incision reinforcing element, b) the absorbable coating, and c) the long-term mesh. This feature is particularly useful in reducing the incidence to re-herniation and erosion.

The implant of claim 1, wherein the incision reinforcement element is planar, and wherein the incision reinforcement element is glued, tied, melted, or otherwise attached to a first side of the mesh. In these embodiments, the flexular modulus of the implant in decreased.

It is a further object to provide an incision reinforcing structure coupled to a mesh structure that is substantially planar on a side of or juxtaposed within the openings of a mesh structure, wherein at least one surface is textured to as to support against opening of an incision line under pressure.

It is a further object to provide an implant comprising an incision reinforcing structure comprising a textured surface. The textured surface is capable of providing a Wenzel state subsequent to implantation. In other embodiments, a Cassie state is created subsequent to implantation. In still other embodiments, the texture provides a mixed Cassie-Wenzel state, or a wettable Cassie state, or a petal effect, or a lotus effect subsequent to implantation, such that the surface prevents opening, tearing or rupture of an incision line. Implantable surface textures are described in U.S. patent application Ser. Nos. 13/745,381 and 13/745,406, which are hereby incorporated by reference in their entirety.

In some embodiments, the incision reinforcing element is an absorbable film, and the implant further comprises a second absorbable film overlying the first absorbable film. In some embodiments, the second absorbable film is preferably laminated to the incision reinforcing film. In some embodiments, the first absorbable film comprises a healing stimulus and the second absorbable film comprises an anti-adhesion aspect, wherein the first film diffuses into the incision defect and promotes joining of the incision surfaces. The second film prevents the incision healing response from coupling to the implant, such that remodeling of the implant due to healthy ingrowth of metabolic tissue on the opposite side is preferably decoupled from the healing aspect of the incision line.

In some embodiments, the absorbable coating is adhesive to the incision-reinforcing element. In this embodiment, the coating can be disposed between the mesh and the incision reinforcing element. For example, the incision reinforcing element may be a planar or sheet like structure, and the coating is disposed on the mesh such that it provides adhesion between the mesh and the reinforcing element. For example the coating could be polydioxanone, disposed between the mesh and the incision reinforcing element, such that the two elements are coupled resiliently for the duration of the intended residency time in vivo.

In some embodiments, the surgical implant further comprises an alignment marker. The alignment marker comprises, in some embodiments, a first marking line preferably extending to a first end of the composite implant. In further embodiments, the alignment marker comprises a second marking line that extends to a second end of the composite implant, and additionally the first and second marking lines may have different widths, which may be used for distinguishing the different sectors of the implant for orienting the implant relative to tissue.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 depicts a surgical implant of the present disclosure in relation to a surgical incision site.

FIG. 2 depicts and implant of the present disclosure comprising a base mesh element, a structural head element, and issue engagement portions.

FIG. 3 depicts a colpopexy and mesh construct of the present disclosure.

FIG. 4 depicts a Y-shaped implant of the present disclosure.

FIG. 5 depicts a structurally reinforced coated mesh, in which the coating bridges the interstitial spaces of the mesh.

FIG. 6 depicts an implant of the present disclosure, further comprising a textured surface layer.

FIG. 7 depicts an anisotropic mechanical mesh in accordance with an embodiment of the present disclosure.

FIG. 8 depicts an implant further comprising an alignment marker.

### DETAILED DESCRIPTION

Reference now will be made in detail to the embodiments of the present disclosure, one or more examples of which are set forth herein below. Each example is provided by way of explanation of the embodiments of the present disclosure and is not a limitation. In fact, it will be apparent to those skilled in the art that various modifications and variations can be made to the teachings of the present disclosure without departing from the scope or spirit of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

The present disclosure provides an implant and a method of repairing an anatomical defect or surgical defect, such as a tissue or muscle wall defect, by promoting tissue growth thereto in a temporally staged fashion. Typically a tissue defect initiates a clinical response in which tissue structures are reinforced by medical devices. To perform the reinforcement procedure a surgical incision must be performed. The incision can be quite minor, as is the case with laparoscopic procedures, but no matter how small the defect created surgically, this defect becomes a locus for stress. So whereas a hernia or prolapse represents a failure of the homogenous structures, either globally or locally, surgical incision focuses these forces to a very small area. Thus, the repair can be more harmful than the initial condition, which is the current experience in certain procedures, such as female pelvic floor repair, where the hernia or prolapse force is directly translated to erosional force.

Current methods of repair are bimodal, e.g., either a permanent or absorbable implant is used, and therefore miss this aspect of the clinical situation. There is first a chronic condition of the prolapse, or generally weakening of tissue due to age or childbirth or prior injury or surgery. There is second a surgical intervention, which is characteristically acute, but nevertheless focuses these chronic forces. Thus, absorbable implants seek to support for a limited time and disappear, but this ignores the chronic condition. The permanent implants, address the chronic condition, but exacerbate the acute surgical intervention by adding an elevated inflammatory response usually associated with cell death, which further weakens focally the surgical intervention site. Lastly, a very large mass of foreign material will typically never be tolerated by the body. Encapsulation of an implant is critically negative to repair of tissue, since the body attacks the encapsulated and tries to remove it from the body. The mechanisms employed in ridding the body of an encapsulated mass can induce apoptosis, release reactive oxygen species, and generally reduce the order and integrity of the surrounding tissue. Thus, repair of tissue, especially tissue close to a surface, such as the vaginal cuff, should integrate with metabolic tissue and be functional or otherwise be vulnerable to exclusion exteriorly.

The present disclosure provides solutions to these clinical and physiological realities. First, an element of the implant in the region of the incision should support the incision post-operatively. Second, a support scaffold can optionally be rendered biocompatible by a coating to support or promote tissue ingrowth or healing. Third, the support scaffold needs to remain after tissue growth and healing to address the chronic aspect that prompted the clinical condition. This staged approach provided herein overcomes the limitations of current implants and methods.

Erosions, mechanical failures, and biocompatibility of the implant can be linked to matching both the chemical and structural aspect of living tissue, and finally providing a mechanism whereby the natural tissue takes over the structural aspect. It is understood that because of age, or extreme trauma, including child birth, the structural distribution of tissue may be so displaced as to require intervention. It is believed that a minimal residual synthetic structure can provide the strength required for repair without chronic inflammatory or foreign body processes.

As used herein “non-biodegradable” or “permanent” regarding an implant mean a material that contains components that are not readily degraded, absorbed, or otherwise reduced when present in living tissue. Such non-biodegradable or permanent materials may be present in living tissue for a period of years, decades, or for the lifetime of the patient in which they are implanted.

As used herein “biodegradable” and “bioabsorbable” regarding implants mean a material that contains components that can be degraded and/or absorbed at some time after implantation of the surgical prosthesis, such as within days, weeks, months, or even several years following implantation.

As used herein “substantially” means predominantly but not wholly that which is specified. For example, when a material is said to be substantially non-biodegradable, it refers to a material that is predominantly composed of non-biodegradable material. When a material is said to be substantially biodegradable, it refers to a material that is predominantly composed of biodegradable material.

A “long-term-stable polymer” or “permanent polymer” refers to a non-absorbable polymer or a very slowly absorbable polymer which still possesses at least 50% of its original tearing strength 60 days after the implantation. Permanent polymers include substances such as e.g. polyamide, polypropylene, polyester, polyurethane, polyether-urethane, which are generally regarded as resistant, as they are not designed as absorbable materials. The long-term mesh described herein comprises a long-term stable or permanent polymer. The long-term mesh is not readily absorbed in vivo, and resides in vivo after implantation well after the incision reinforcing element and absorbable coating have been absorbed. Thus, the long-term mesh is permanent or substantially permanent in vivo, and is able to last for a period of many years, or decades, or even longer.

The implant of the present disclosure comprises a long-term-stable or long-term mesh or mesh-like basic structure with pores. In certain embodiments, the total area of the pore structure comprises more than 90% of the total area of the mesh, and the diameter of the pores lies in the range from about 1 mm to about 8 mm.

Materials useful for the mesh element of the implants disclosed herein include polypropylene, polyester, polyurethane or more exotic forms such as halogen polymers such as mixtures of polyvinylidene fluoride and copolymers of vinylidene fluoride and hexafluoropropene. Other materials are also conceivable. In certain embodiments, the material used for the mesh is a long-term stable polymer, such as a polypropylene, polyether, or polyurethane. In certain embodiments, the mesh further comprises a biocompatible and bioabsorbable coating.

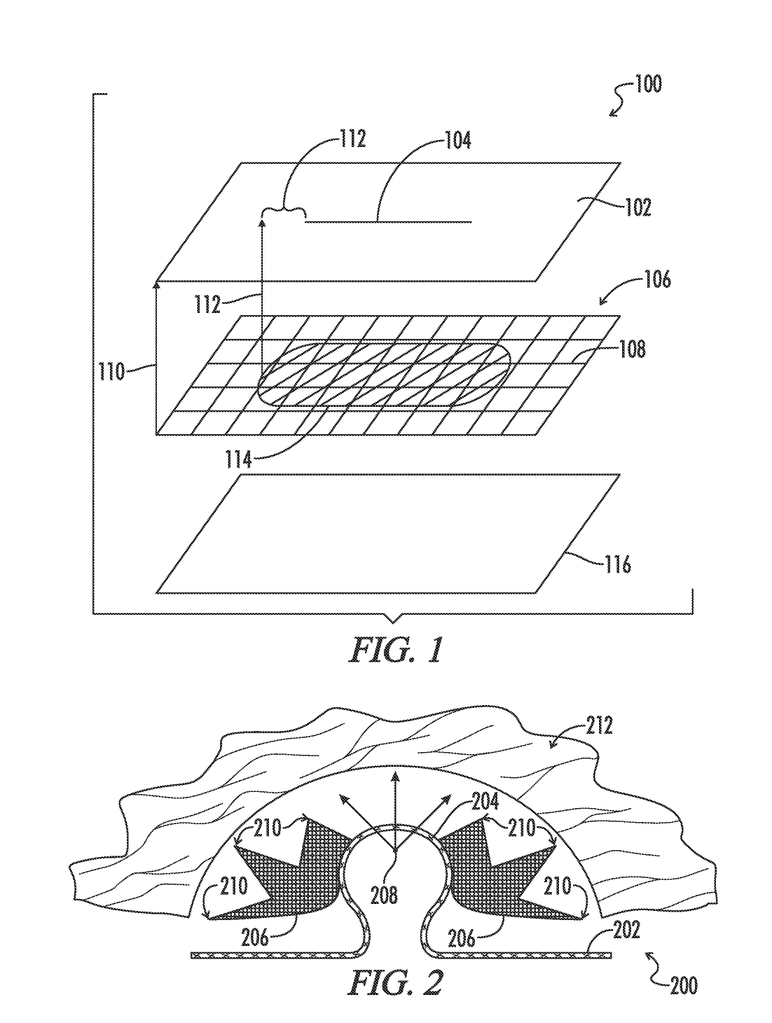

The implants further comprise an incision reinforcement element. In some embodiments, the incision reinforcement element comprises a layered or sheet-like structure disposed on or in communication with the mesh element. For example, FIG. 1 illustrates an embodiment of the present invention in relation to a surgical incision 100. Plane 102 is a biological tissue surface through which an incision 104 is made. Directly beneath tissue plane 102 is the surgical implant 106. Arrows 110 and 112 make the plane projection of mesh 108 onto tissue plane 102, such that it is apparent that reinforcement element 114 is directly below incision 104 and extends beyond incision 104 by an additional distance 116. Accordingly, the weakness in tissue layer 102 caused by incision 104 is prevented from herniation of internal tissue 116 situated below mesh 108 by the reinforcement element 114. Particularly suitable materials for the reinforcing element are poly-p-dioxanone, lactide polymers, copolymers of glycolide and lactide (e.g. in the ratio 9:1) and mixtures of poly-p-dioxanone and polyethylene glycol, various absorbable polyurethanes, and other synthetic, absorbable materials are also possible.

The incision reinforcement element in some embodiments may comprise a gel, foam, film or membrane made of a bioresorbable material. The incision reinforcement element may be prepared from one or more components selected from hyaluronic acids and any of its salts, carboxymethyl cellulose and any of its salts, oxidized regenerated cellulose, collagen, gelatin, phospholipids, and various d and l forms of polylactic acid, as well as any cross-linked or derivatized forms thereof. In some embodiments, the barrier is made from a material capable of forming a hydrogel when contacted with an aqueous fluid, such as saline, phosphate buffer, or a bodily fluid.

The incision reinforcement element is bioabsorbable in vivo. In certain embodiments, the incision reinforcement element is bioabsorbed over a time period similar to the time needed for healing of the incision cite. For example, in some embodiments, the incision reinforcement element is absorbed over a period of days or up to several weeks. For example, the period of absorption may be about 1 day to about 4 weeks, about 1 day to about 2 weeks, about 1 day about 7 days, or about 2 to about 7 days.

In one embodiment, the incision reinforcement composition comprises a mixture of at least two polymer systems. The first polymer system includes a cross-linked biodegradable multi-block polymer hydrogel having a three-dimensional polymer network. The second polymer system comprises a polylactic acid polymer.

The absorbable mesh coating comprises, in some embodiments, a biocompatible absorbable polymer. In some embodiments, the coating is cross-linked on the mesh structure. Generally the underlying mesh structure is strong in terms of tensile strength, but also generally relatively fibrogenic. A common example is polypropylene. In order to render the polypropylene more biocompatible. It can be coated with a hydrophilic polymer, preferably a polymer hydrogel comprised of hydrophilic blocks, biodegradable blocks, and crosslinking blocks formed during the polymerization on the mesh. One or more of these blocks may themselves be polymeric in nature.

Suitable hydrophilic polymeric blocks include those which, prior to incorporation into the macromer, are water-soluble such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, hydroxyalkylated celluloses such as hydroxyethyl cellulose and methylhydroxypropyl cellulose, polypeptides, polynucleotides, polysaccharides or carbohydrates such as polysucrose, hyaluronic acid, dextran, heparin sulfate, chondroitin sulfate, heparin, or alginate, and proteins such as gelatin, collagen, albumin, or ovalbumin. The preferred hydrophilic polymeric blocks are derived from poly(ethylene glycol) and poly(ethylene oxide).

The biodegradable blocks are preferably hydrolyzable under in vivo conditions. Biodegradable blocks can include polymers and oligomers of hydroxy acids, carbonates or other biologically degradable polymers that yield materials that are non-toxic or present as normal metabolites in the body. Preferred oligomers or polymers of hydroxy acids are poly(glycolic acid), also called polyglycolate, poly(DL-lactic acid) and poly(L-lactic acid), also called polylactate. Other useful materials include poly(amino acids), poly(anhydrides), poly(orthoesters), and poly(phosphoesters). Polylactones such as poly(epsilon-caprolactone), poly(delta-valerolactone), poly(gamma-butyrolactone) and poly(beta-hydroxybutyrate), for example, are also useful. Preferred carbonates are derived from the cyclic carbonates, which can react with hydroxy-terminated polymers without release of water. Suitable carbonates are derived from ethylene carbonate (1,3-dioxolan-2-one), propylene carbonate (4-methyl-1,3-dioxolan-2-one), trimethylene carbonate (1,3-dioxan-2-one) and tetramethylene carbonate (1,3-dioxepan-2-one).

Polymerizable groups are reactive functional groups that have the capacity to form additional covalent bonds resulting in macromer interlinking. Polymerizable groups specifically include groups capable of polymerizing via free radical polymerization and groups capable of polymerizing via cationic or heterolytic polymerization. Suitable groups include, but are not limited to, ethylenically or acetylenically unsaturated groups, isocyanates, epoxides (oxiranes), sulfhydryls, succinimides, maleimides, amines, imines, amides, carboxylic acids, sulfonic acids and phosphate groups. Ethylenically unsaturated groups include vinyl groups such as vinyl ethers, N-vinyl amides, allyl groups, unsaturated monocarboxylic acids or their esters or amides, unsaturated dicarboxylic acids or their esters or amides, and unsaturated tricarboxylic acids or their esters or amides. Unsaturated monocarboxylic acids include acrylic acid, methacrylic acid and crotonic acid or their esters or amides. Unsaturated dicarboxylic acids include maleic, fumaric, itaconic, mesaconic or citraconic acid or their esters or amides. Unsaturated tricarboxylic acids include aconitic acid or their esters or amides. Polymerizable groups may also be derivatives of such materials, such as acrylamide, N-isopropylacrylamide, hydroxyethylacrylate, hydroxyethylmethacrylate, and analogous vinyl and allyl compounds.

The polymerizable groups are preferably located at one or more ends of the macromere, such that when placed on a mesh structure as a prepolymer it begins to polymerize around the mesh structure. The polymerization may be within the coating composition or additionally between the coating composition and the underlying mesh structure.

At least a portion of the macromers may contain more than one reactive group per molecule so that the resulting hydrophilic polymer can be crosslinked to form a gel. The minimal proportion of crosslinkers required will vary depending on the desired properties of the hydrogel to be formed and the initial macromer concentration in solution. The proportion of crosslinkers in the macromer solution can be as high as about 100% of all macromers in the solution. For example, the macromers include at least 2.5 polymerizable groups on average, and, more preferably, the macromers each include three or more polymerizable groups on average. Poloxamines, an example of water-soluble polymer component suitable to form a hydrophilic block, have four arms and thus may readily be modified to include four polymerizable groups.

In some embodiments, the coating is absorbed in vivo over a period of weeks, months, or even up to about a year. For example, the coating may absorbed in vivo after about a week to about one year, about a week to about 6 months, a week to about 3 months, or about a week to about one month. In other embodiments, the

In certain embodiments, the implants according to the present disclosure are characterized in that tissue grows surprisingly quickly and well into the implant. This is likely due to the inventive feature of addressing the acute aspects acutely, and providing for natural tissue healing chronically, wherein a minor reinforcement scaffold remains.

There is also a little appreciated aspect of absorbable implants, wherein they tend to lose their mechanical strength first and their inflammation-inducing mass secondarily. For example, the actual absorption of the polymers of absorbable films can last for months, while the integrity and the stability of the films is already reduced after less than 4 weeks. Even more worrisome, is that the clinically preferred materials decompose into small fragments. Clinically the focus has been on molecular biocompatibility, but the presence of fragments in the body can be inflammatory apart from the biocompatibility aspects of the molecular structure.

However, contrary to teachings elsewhere, tissue integration and polymer decomposition cannot be decoupled. In particular, positive and negative aspects of tissue response such as adhesions, ingrowth, neovascularization, encapsulation, etc. tend to possess characteristic evolution times, wherein after a characteristic period they do not occur. Thus, appreciating this aspect is useful to achieving wound-healing and the development of a new tissue layer in and over the implant.

Alternatively, the disclosure may be viewed as a surgical implant for treating a pelvic floor disorder, such as for a sacral colpopexy procedure. The implant comprises a base mesh element, the structure of which may be shaped, and possessing a head portion comprising two tissue engagement portions extending from the base portion, and separation force distribution element for attaching at least one of the tissue engagement portions to the base portion in a fashion that distributes a force that would tend to separate a tissue engagement portion from the base portion across an areal region greater than that occupied by a suture. The separation force distribution element may comprise a central strengthening element and a tissue gripping element. An example is illustrated in FIG. 2, which shows a side view of an implant 200 comprising a base mesh element 202, which forms a structural head element 204, the combination of 202 and 204 comprising two tissue engagement portions 206 extending from the head element 206, wherein tissue engagement portions 206 distribute forces 208 into tissue engagement sections 210 which are distributed on an areal tissue surface 212.

The disclosure also relates to implants for treating pelvic organ prolapse, comprising a step of making an incision in a wall of the vagina to form a space between the vagina and an organ to be supported.

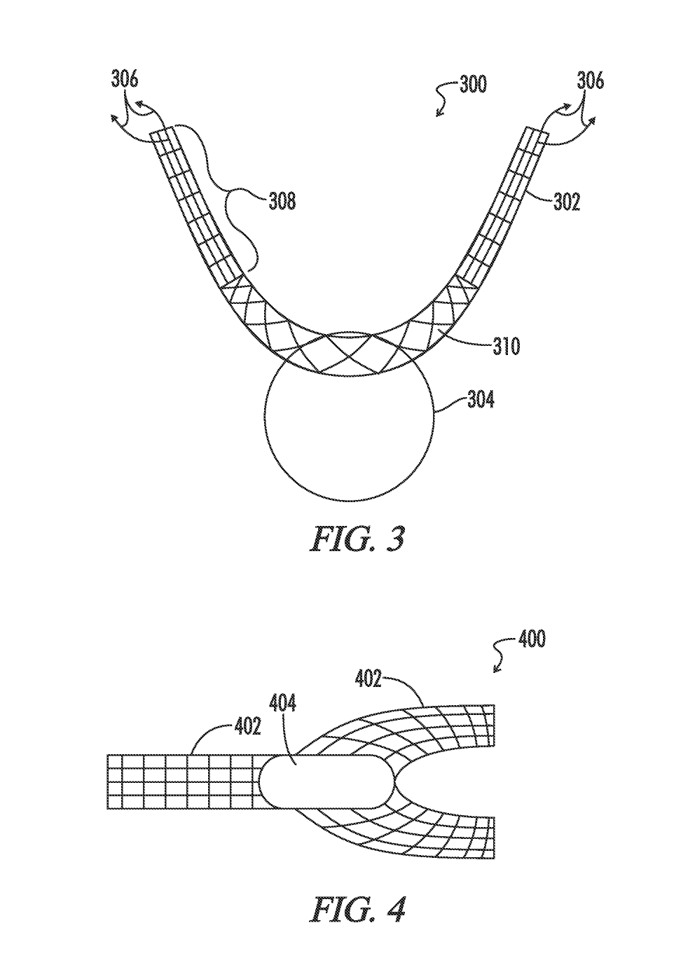

The implant of the present disclosure in certain embodiments comprises a structural layer, a mesh and a mesh coating. In one embodiment, the mesh comprises a polymeric material or fabric and the coating comprises a crosslinkable species. After coating, the surgical mesh preferably remains porous, to afford tissue ingrowth. The coating can be used to affix the structural layer to the mesh. Preferably, if the implantable article is to be used in a sacral colpopexy procedure, the implantable article is sized and shaped to loosely extend from the patient's sacrum to the patient's vagina with at least some slack. The colpopexy and mesh construct 300 are depicted in FIG. 3. Mesh 302 and vaginal cuff 304 are depicted in the typical sling arrangement. Arrows 306 indicate pelvic attachment. Porous region 308 affords tissue ingrowth. Structural layer 310 provides areal support and mitigate against tissue erosion.

Alternatively, portions of the implantable article (e.g. the mesh portion) may comprise suture bridges instead of fabric or substantially flat, planar structures.

The composite surgical implant in some embodiments preassembled in a T-shape or Y-shape and is sterile packaged, in addition to more typical square, rectangle, circle, or oval shape. For example, the implant may comprise a sheet-like structure of any shape or in strips. The sheet structure is comprised of an incision reinforcing element, which can be in the form a film, sheet or layer, in communication with the mesh. For example, the absorbable coating may be disposed between the mesh and the incision reinforcing element. In other embodiments, the mesh is coating with the absorbable coating, and the incision reinforcing element is glued, tied, melted, or otherwise affixed to the coated mesh. In these and other embodiments, the absorbable coating may be disposed with in the openings of the mesh, and thus be substantially coplanar with the mesh. In yet another embodiment, the incision reinforcement element is cast within the openings of the mesh such that the incision reinforcement element lies substantially coplanar with the mesh. FIG. 4 illustrates a Y-shaped embodiment comprising a mesh layer 402 and an incision reinforcing element 404. A T-shaped arrangement is also possible.

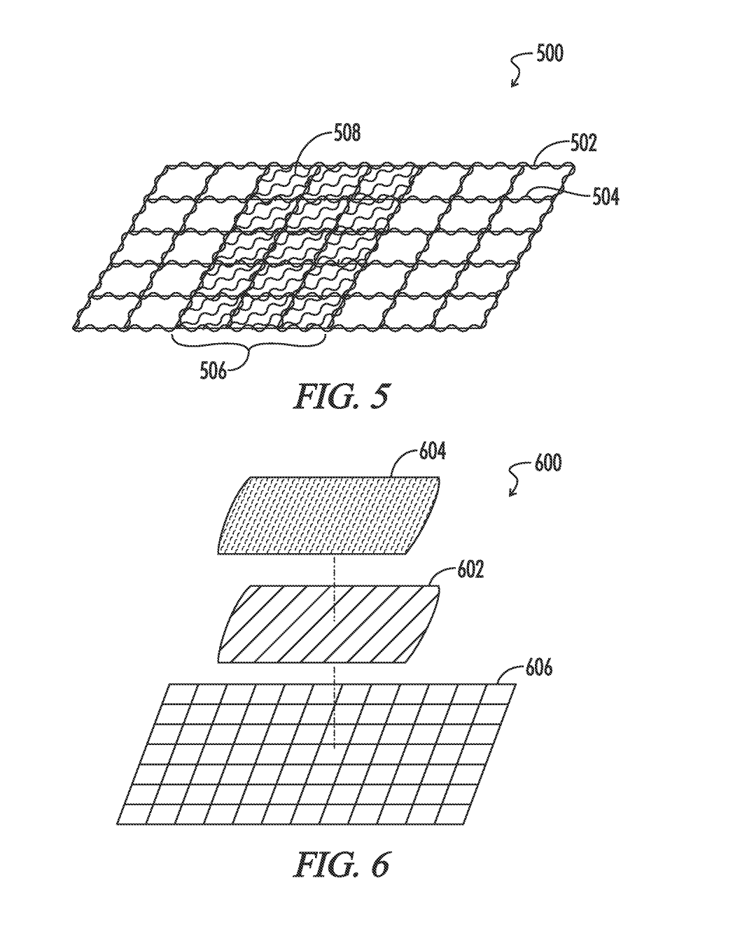

FIG. 5 illustrates a structurally reinforced coated mesh 500 with mesh elements 502 and coating 504. In a reinforcing locus 506, the coating 504 bridges intersticial spaces of the mesh 508 to produce a planar element in the plane of mesh elements 502.

The implant of the present disclosure may have the following characteristics, as depicted in FIG. 6: the incision supporting element 602 comprises a layer 604 with a textured surface disposed on a mesh element 606 to help hold together an incision line. The overall contour of the device globally can be of general oval or elliptical shape, though other shapes as described above may be used. The implant comprises a soft mesh of high compliance, a coating to hold the mesh in an intended configuration, and incision reinforcement layer to reduce aneurization of an incision line. In some embodiments, the incision reinforcement element is essentially coextensive with the underlying mesh and mesh coating. In other embodiments, as shown in FIG. 6, the incision reinforcement layer 602 has a smaller area than the mesh 606, and may further be shaped in accordance with the desired surgical procedure. For example, the incision reinforcement element may be in the shape of the anticipated incision line or laparoscopic entry point, or otherwise shaped to provide the need short-term structural support.

A soft mesh useful in the present implant comprises a web comprised of interlaced fibers. The fibers are comprised of polypropylene, polyester or a material of animal or human origin. The incision bracing layer can be of a folded or corrugated geometry, which may additionally allow insertion of the implant in the tissues as far as a working position and be able to be deployed when the implant reaches the working position.

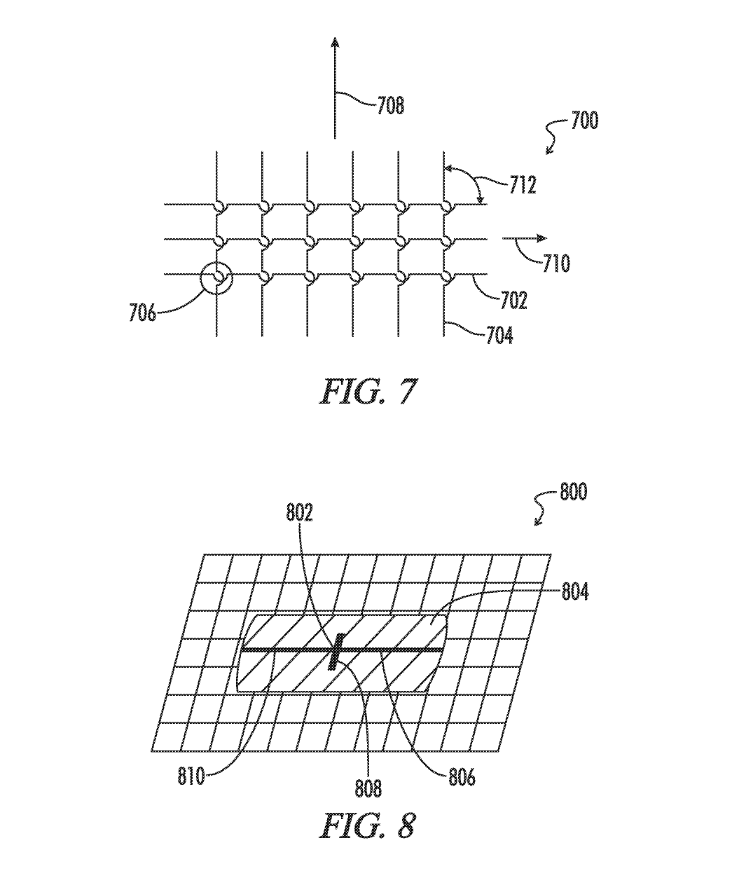

In one embodiment, a composite tissue reinforcing implant preferably includes a base component, such as a surgical mesh. In one embodiment, the surgical mesh preferably has anisotropic mechanical properties so that the mesh is more stretchable in a first direction and less stretchable in a second direction, and a spectrum of stretchable characteristics along predetermined directions. For example, in an embodiment, the mesh has a distension anisotropy that lies on two more axes, and comprises any combination of angles. Thus, after bioabsorbtion of the absorbable coating and the incision reinforcement element, the mesh structure is more distensible in one direction than the other FIG. 7 illustrates an anisotropic mechanical mesh 700 wherein there are horizontal mesh fibers 702 and perpendicular mesh fiber 704, as is typical. However, the spacing of knots 706 dictates the distensibility in directions 702 and 704. The knot density is higher in the perpendicular bdirection and the distension per unit force is given by the magnitude of vector 708. The density of knots in the horizontal direction is higher, and hence the distensibility is low as indicated by the magnitude of vector 710. The angle 712 can vary, and need not be orthogonal.

The areal implant according to the disclosure has a flexible basic structure made from a fabric or stamped planar constitution, comprising non-resorbable material or resorbable material or a combination of such materials. If resorbable material is used, the resorption time (i.e. the period after which the total mass of the implant has degraded in vivo) is at least variable based on the physiology, and/or in other circumstances the in vivo decrease in strength is more relevant than mass loss, whereby such considerations are appreciated.

Non-resorbable or slowly resorbable materials are used in order that the basic structure is stable in the longer term and augment of native tissue is required long term to ensure healing success.

Knitted fabric of the basic persistent structure can be designed to stretch more than the tissue region destined to receive the implant below a critical force and stretch less than this tissue region above a critical force. The critical force is below the highest load to which this tissue region can be submitted without harm. The flexible, basic structure is preferably matched to the usual movements of the tissue (e.g. of an abdominal wall) into which the areal implant is inserted or sewn. In the case of small forces, as occur during normal movements by the patient, the elasticity behavior of the implant matches that of the abdominal wall and the inserted implant is shaped by the abdominal wall. The implant thus does not act as a foreign body in its mechanical aspects. If, on the other hand, the forces exceed the critical force, the implant absorbs the forces and thus prevents injury to the body tissue, e.g. the abdominal wall.

According to the disclosure, the basic structure is stiffened by a synthetic absorbable material whose absorption time is variable and coincident with physiological processes. For example, for incision repair typically the absorption time can be 3 to 7 days and for promoting vascularized tissue ingrowth and prohibition of adhesion typically the absorption time can be 7 day to several months, and for chronic support of tissue the mesh structure may be substantially non-absorbable. In particular a layer is placed on the mesh structure in the vicinity of the surgical incision to locally strengthen the region, a coating is placed on the mesh structure to reduce inflammation and promote healthy tissue ingrowth. Patient age also plays a role, in older patients healing processes may be delayed. As a result, the areal implant is relatively firm and easy to handle during the operation (e.g. when cutting to size and inserting) and loses its rigidity after a relatively longer time in the body tissue than had it otherwise been placed in younger or healthier tissue.

A composite implantable device for promoting tissue ingrowth therein is provided, comprising (i) a first biodurable reticulated elastomeric matrix having a two-dimensional porous structure comprising a continuous network of interconnected and intercommunicating open pores, (ii) a polymeric coating comprising a basically hydrophilic surface, and iii) an incision structural element.

Embodiments may also include one or more of the following features. The incision support of the surgical implant includes polymer hydrogel. The coating barrier may include a polyanionic polysaccharide modified by reaction with carbodiimide. In some embodiments, the coating adhesion includes a crosslinked polymer hydrogel alone or in combination with at least one polyanionic polysaccharide modified by reaction with carbodiimide or isocyanate. The crosslinked polymer hydrogel includes one or more hydrophilic blocks, one or more biodegradable blocks, and one or more crosslinking blocks. The crosslinked polymer hydrogel is formed by polymerization of monomers including photopolymerizable poly(ethylene glycol)-trimethlyene carbonate/lactate multi-block polymers endcapped with acrylate esters. The polyanionic polysaccharide modified by reaction with carbodiimide/isocyanate includes carbodiimide/isocyanate-modified hyaluronic acid and carbodiimide/isocyanate-modified carboxymethylcellulose.

Embodiments may also include one or more of the following. The mesh coating or the incision support layer includes a crosslinked polymer hydrogel comprising a hyaluronan and a polyurethane group. The crosslinked polymer hydrogel includes one or more hydrophilic blocks, one or more biodegradable blocks, and one or more crosslinking blocks. In some embodiments, the crosslinked polymer hydrogel is formed by polymerization of monomers including hyaluronan with ether groups via carbamate or urea links.

In further embodiments, such as an embodiment depicted in FIG. 8, the implant 800 comprises an alignment marker 802 adapted to be positioned at a center of the incision reinforcing element 804, and coincident with an incision line, wherein a first marking line 806 extends from a first side of the central region 808 of the alignment marker, and a second marking line 810 aligned with the first marking line extends from a second side of the central region of the alignment marker. In certain embodiments, the alignment marker is located in a central region of a surface of the implant, while in other embodiments, the alignment marker be in other areas of the implant surface. The alignment marker can vary depending on the shape of the implant and specific surgical use. The first and second marking lines are preferably aligned with the distensible axis of the anisotropic mesh, such that when marker lines are aligned parallel with an incision line, minimal distention occurs perpendicular and in plane with the incision. In still further embodiments, the alignment marker can be disposed on the mesh. In further embodiments, an absorbable film overlies the alignment marker

The devices and compositions of the present disclosure may be free of substantially free of any optional or selected ingredients described herein. In this context, and unless otherwise specified, the term “substantially free” means that the selected item may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in implantable medical devices.

As used herein, the term “about” should be construed to refer to both of the numbers specified in any range. Any reference to a range should be considered as providing support for any subset within that range.

Examples are provided to illustrate some embodiments of the embodiments of the present disclosure but should not be interpreted as any limitation thereon. Other embodiments within the scope of the claims herein will be apparent to one skilled in the art from the consideration of the specification or practice of the embodiments or methods disclosed herein. It is intended that the specification, together with the example, be considered to be exemplary only, with the scope and spirit of the disclosure being indicated by the claims which follow the example.

What is claimed is:

1. A surgical implant comprising a bioabsorbable incision reinforcement element, a long-term mesh, and a bioabsorbable coating disposed on the mesh.

2. The surgical implant of claim 1, wherein the incision reinforcement element comprises a bioabsorbable material that degrades during a first time period, and the coating comprising a bioabsorbable material that degrades in a second time period, and the first time period is shorter than the second time period.

3. The surgical implant of claim 1, wherein the long-term mesh comprises an areal mass of less than about 30 g/m².

4. The surgical implant of claim 3, wherein the long-term mesh comprises an areal mass of less than about 15 g/m².

5. The surgical implant of claim 1, wherein the bioabsorbable coating persists in vivo for a period of time between about 7 days and about 2 years.

6. The surgical implant of claim 1, further comprising an alignment marker.

7. The surgical implant of claim 1, wherein the incision reinforcement element is planar, and wherein the incision reinforcement element is glued, tied, melted, or otherwise attached to a first side of the mesh.

8. The surgical implant of claim 1, wherein the incision reinforcement element is planar, and wherein the incision reinforcement element is cast within the openings of the mesh such that the incision reinforcement element lies substantially coplanar with the mesh.

9. The surgical implant of claim 1, wherein the mesh comprises a spatially variable distensibility that changes temporally, wherein a first region underlying a surgical incision line has substantially no distensibility upon implantation, and wherein a second bioabsorbable coating extends over the majority of the implant.

10. The surgical implant of claim 1, wherein the second absorbable coating is absorbable during a period of time longer than the second time period.

11. The surgical implant of claim 1, wherein the mesh has a distension anisotropy that lies on two or more axes.

12. The surgical implant of claim 11, wherein the mesh has an orthogonal or substantially orthogonal distension anisotropy.

13. The surgical implant of claim 1, wherein initially upon implantation in vivo, the implant has minimal distensibility in a line perpendicular to and planar to a surgical incision in at least one of a) the incision reinforcing element, b) the bioabsorbable coating, and c) the long-term mesh.

14. The surgical implant of claim 1, wherein the incision reinforcing element is substantially planar and is disposed on a side of the mesh or is disposed within the openings of the mesh, wherein the incision reinforcing element comprises a textured surface, and wherein the textured surface provides a Wenzel state, a mixed Cassie-Wenzel state, or a wettable Cassie state, or a petal effect, or a lotus effect subsequent to implantation.

15. The surgical implant of claim 1, wherein the incision reinforcing element comprises a first absorbable film, and a second absorbable film overlies the first absorbable film.

16. The surgical implant of claim 16, wherein the second absorbable film comprises a healing stimulus and an adhesion aspect,
such that upon implantation in vivo, the first absorbable film diffuses into an incision site, thereby promoting joining of incision surfaces, and
the second absorbable film prevents the incision site from coupling to the implant.

17. The surgical implant of claim 1, wherein the absorbable coating is adhesive to the incision reinforcing element such that the absorbable coating couples the incision reinforcing element to the mesh.

18. The surgical implant of claim 1, wherein the long-term mesh comprises a flexible knitted fabric comprising a material selected from the group consisting of polypropylene, polyester, polyurethane, polyvinylidene fluoride and copolymers of vinylidene fluoride, hexafluoropropene, or mixtures thereof.

19. The surgical implant of claim 1, wherein the absorbable coating comprises a material selected from the group consisting of polycaprolactone, polyglycolide, polylactide, poly-p-dioxanone, lactide/glycolide copolymers, lactide/caprolactone copolymers, glycolide/caprolactone copolymers, glycolide/poly-p-dioxanone copolymers, lactide/caprolactone copolymers, glycolide/caprolactone copolymers, glycolide/poly-p-dioxanone copolymers, glycolide/poly-p-dioxanone/lactide copolymers, and mixtures thereof.

20. The surgical implant of claim 1, wherein the incision reinforcing element comprises a gel, foam, film or membrane comprising a bioabsorbable material comprising hyaluronic acids or any of its salts, carboxymethyl cellulose or any of its salts, oxidized regenerated cellulose, collagen, gelatin, phospholipids, polylactic acid, poly-p-dioxanone, lactide polymers, copolymers of glycolide and lactide, mixtures of poly-p-dioxanone and polyethylene glycol, absorbable polyurethanes, or any mixture thereof.
